(19) United States
(12) Patent Application Publication
(10) Pub. No.: US 2024/0390346 A1
(43) Pub. Date: Nov. 28, 2024

(54) COMPOSITIONS FOR A DIETARY SUPPLEMENT TO MANAGE SENESCENT CELLS

(71) Applicant: Neurohacker Collective, LLC, Carlsbad, CA (US)

(72) Inventor: Gregory Kelly, Oceanside, CA (US)

(21) Appl. No.: 18/202,051

(22) Filed: May 25, 2023

(51) Int. Cl. A61K 31/4412; A61K 31/12; A61K 31/352; A61K 31/357; A61K 36/22; A61K 36/258; A61K 36/28; A61K 36/48; A61K 36/489; A61K 36/63; A61K 36/67; A61K 36/9066; A61P 39/00

(52) U.S. Cl. CPC A61K 31/4412; A61K 31/12; A61K 31/352; A61K 31/357; A61K 36/22; A61K 36/258; A61K 36/28; A61K 36/48; A61K 36/489; A61K 36/63; A61K 36/67; A61K 36/9066; A61P 39/00

(57) ABSTRACT

A composition and method for improving mental function and physical performance with aging is disclosed. The composition comprises Rhus succedanea stem extract standardized for Fisetin, Piper longum root extract standardized for Piperlongumine, and Sophora japonica L. flower extract standardized for Quercetin. The composition may optionally include Sophora japonica L. flower extract standardized for Luteolin, Curcuma longa root standardized for curcuminoids, Olive Leaf extract standardized for olive polyphenols, milk thistle seed extract standardized for silymarin, soybean seed extract standardized for soy isoflavones, and Senactive® senolytic or combinations thereof. The composition is formulated for oral administration and may be taken intermittently for a couple to a few days at a time, followed by a week to several weeks without supplementation, with this dosing regimen repeated.

### BACKGROUND OF THE INVENTION

### 1. Field of Invention

The present invention relates to dietary supplements and methods of managing senescent cells to improve mental function and physical performance during aging.

### 2. Description of Related Art

The population of the United States, and other developing countries is aging—the number of Americans ages 65 and older is expected to more than double over the next 40 years. There is a growing desire among adults of all ages, especially middle-age and older adults, in maintaining mental function and physical performance throughout the lifespan.

Cellular senescence is one of the nine Hallmarks of Aging (López-Otín et al., 2013). It refers to the gradual accumulation of senescent cells in tissues throughout the body during aging. Senescent cells are cells that have stopped dividing but have not been removed, and which express stereotyped phenotypic changes. One of these phenotypic changes is the senescence associated secretory phenotype (SASP). Cells become senescent in response to a variety of factors including telomere shortening, molecular damage, mitochondrial dysfunction, and cellular stress.

Senescent cells accumulate during aging because of three main reasons: (1) lingering senescence cells exploit networks of senescent cell anti-apoptotic pathways (SCAPs) that prevent them from proceeding through apoptosis, (2) an aging immune system (immunosenescence) is less able to detect and eliminate senescent cells, and (3) senescent cells, because of the SASP they secrete into their environment, can cause nearby non-senescent cells to become senescent (i.e., secondary senescence).

Senescent cells accumulate in tissues during aging. This accumulation can contribute to many age-related diseases and pathologies. These include autoimmunity, cardiovascular disease, chronic obstructive pulmonary disease (COPD), diabetes, kidney disease, macular degeneration, neurodegenerative diseases, and osteoarthritis. Senolytic drugs are being developed with the hope that they may eventually be used to treat age-related diseases and pathologies.

There remains an unmet need for compositions and methods for reducing symptoms of aging. More particularly, it would be a boon to the aging population to develop safe and effective botanically-derived supplements which improve mental function and physical performance for individuals in need thereof.

### SUMMARY OF THE INVENTION

The present invention is directed to a dietary supplement using a combination of plant extracts designed to support the management of senescence cells in multiple tissues using a variety of SCAPs for survival that is intermittently dosed in order to promote improved mental function and physical performance with aging. The advantages of the invention for managing senescent cells may be experienced in a variety of areas of mental function and physical performance including but not limited to cognitive performance (e.g., focus, mood, motivation, memory, sleep) and physical performance (e.g., energy, flexibility, joint comfort, muscle size and strength, post-exercise recovery).

In one embodiment of the invention, a dietary supplement composition comprises: Rhus succedanea Stem Extract (or suitable other starting material from this or other plants) standardized for Fisetin, Piper longum Root Extract (or suitable starting material from other Piper sp.) standardized for Piperlongumine, and Sophora japonica L. Flower Extract (or suitable other starting material from this or other plants) standardized for Quercetin. In another embodiment of the present invention, the composition further comprises Sophora japonica L. Flower Extract (or suitable other starting material from this or other plants) standardized for Luteolin, Curcuma longa Root standardized for curcuminoids, and Olive Extract (from leaf, stem or their combination) standardized for olive polyphenols, or any combination thereof. In another embodiment of the invention, the composition further comprises Milk Thistle Seed Extract standardized for silymarin, Soybean Extract standardized for soy isoflavones, and Senactiv® (a proprietary combination of Panax notoginseng Root and Rosa roxburghii Fruit Extracts), or any combination thereof. The individual ingredients, or any combination thereof, may or may not be produced in a manner to be more bioavailable.

In an exemplary embodiment of the invention, the composition is integrated into a capsule, tablet, chewable tablet, or pill to be taken orally, which is taken intermittently. The intermittent dosing consists of taking the composition for two to four days, followed by twelve days to thirteen weeks where the composition is not taken. This intermittent dosing protocol is then repeated.

In one aspect, a composition for managing senescent cells is provided. The composition advantageously includes an effective, synergistic amount of Fisetin, piperlongumine, and quercetin. The Fisetin is standardized from Rhus succedanea stem extra, the piperlongumine is derived from Piper longum root extract, the quercetin is derived from Sophora japonica L. flower extract.

Optionally, the composition may further include an effective amount of a botanical agent selected from the group consisting of luteolin sourced from Sophora japonica L. flower extract, curcuminoid sourced from Curcuma longa root, polyphenols sourced from olive leaf extract, and combinations thereof. The composition may also have an effective amount of a silymarin, soy isoflavones, Senactive® senolytic supplement (NuLivScience, Brea, CA), and combinations thereof.

In one aspect, the silymarin is sourced from milk thistle seed extract. In another aspect, the isoflavones are sourced from soybean extracts.

The composition of having botanical active agents may be integrated into a capsule, tablet, chewable tablet, or pill for oral delivery.

The composition may be formulated for a prescribed administration protocol. For example, the administration protocol comprises administration of said composition once daily for a period of at least two days but not more than four days followed by a period of at least twelve days but no more than thirteen weeks without treatment, with the administration repeated following this same dosing protocol.

The composition may be integrated into a liquid or powder.

In yet another aspect of the invention, the effective amount of Fisetin is between about 700 mg to 2500 mg per dose. Optionally, the amount of Fisetin is 1400 mg. The effective amount of quercetin is between about 250 mg and 1500 mg per dose

Also described is a method for improving mental function and physical performance associated with aging. The method includes administering to an individual in need thereof a composition having:
an effective amount of Rhus succedanea stem extract;
an effective amount of Piper longum root extract; and
an effective amount of Sophora japonica L. flower extract;
wherein the administration of said composition manages senescent cells.

The method may further include administering Luteolin, curcuminoids, olive polyphenols, and combinations thereof. Milk Thistle Seed extract, Soybean extracts, Panax notoginseng root extract, Rosa roxburghii fruit extract, and combinations thereof may likewise be administered to improve mental function and physical performance.

The method may include orally administering the composition in the form of a capsule, tablet, chewable tablet, liquid, powder, or pill. It may be administered once a day for a period of two days followed by a period of four weeks without administration of said composition.

The advantages of the invention for managing senescent cells may be experienced in a variety of areas of mental function and physical performance including but not limited to cognitive performance (e.g., focus, mood, motivation, memory, sleep) and physical performance (e.g., energy, flexibility, joint comfort, muscle size and strength, post-exercise recovery).

The foregoing, and other features and advantages of the invention, will be apparent from the following, more particular description of the preferred embodiments of the invention, and the accompanying drawings.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

For a more complete understanding of the present invention, the objects and advantages thereof, reference is now made to the ensuing descriptions taken in connection with the accompanying drawings briefly described as follows:

FIG. 1 illustrates a composition according to an embodiment of the invention;

FIG. 2 illustrates a table of exemplary amounts of ingredients, all of various subsets of which are found in one or more embodiments of the present invention; and

FIG. 3 illustrates a table of exemplary amounts of ingredients, all of various subsets of which are found in one or more embodiments of the present invention.

FIG. 4 illustrates a composition according to an embodiment of the invention used in two examples.

### DETAILED DESCRIPTION OF THE EMBODIMENTS

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

Implementations of the technology described herein are directed generally to dietary supplements for managing senescent cells, particularly in the context of aging. As described above, the accumulation of senescent cells occurs in tissue during aging. While this accumulation has been associated with many age-related diseases, conditions, and pathologies, t senescent cells can also accumulate below the threshold for causing disease and may still contribute to suboptimal mental function and physical function.

As examples, senescent brain cells can hinder cognitive performance, senescent muscle cells contribute to anabolic resistance (i.e., lesser responses to anabolic behaviors and signals), senescent joint cells can make it more challenging to comfortably perform activities of daily living, and senescent skin cells impact visible signs of skin aging such as fine lines and wrinkles. While in these cases there may be no pathology or disease state, the burden of senescent cells being carried in tissues as we get older can make it more challenging to enjoy the same degree of mental function and physical performance experienced at younger ages. Therefore, managing senescent cells well in advance of age-related pathology or disease is an unmet need and a need unlikely to be met by the medical system.

Selected plant extracts that qualify as dietary supplements can support certain aspects of managing senescent cells. In order to produce more optimal mental function and physical performance impact, a specific synergistic combination of plant extracts (i.e., a poly-extract formulation) is provided which is informed by the observation that senescent cells are heterogeneous.

Senescent cells in different tissues are heterogeneous because of their site of origin. The result is that populations of senescent cells in different tissues are not all equally susceptible to being managed by the same plant extracts. Single plant extracts may be successful in some tissues (bone marrow as an example), but less active or ineffective in others (fat tissue as an example). This tissue heterogeneity makes it unlikely that a single plant extract will effectively manage all senescent cells.

Senescent cells are heterogeneous because of the diversity of mechanisms they use for survival. Senescent cells use networks of interacting senescent cell anti-apoptosis pathways (SCAPs) to survive. Because a variety of factors (e.g., cellular stress, mitochondrial dysfunction, telomere shortening) cause cells to become senescent, there can be a great deal of diversity in the types of SCAPs that populations of senescent cells rely on to maintain survival. Single plant extracts may be successful in managing senescent cells that rely on certain SCAPs, but be less active or ineffective in senescent cells using different SCAPs for survival. This mechanism heterogeneity makes it unlikely that a single plant extract, or non-optimized combinations of plant extracts, will effectively manage all senescent cells.

In order to manage senescent cells, as a means of improving mental function and physical performance, the doses of individual plant extracts used within an overall composition are advantageously within certain dosage ranges. This is because sufficient amounts of the active compounds are preferred to interact with senescent cells and effectively manage them: lower doses would not be expected to optimally manage senescent cells.

Dietary supplements are typically dosed every day, often with a recommendation to take a dose more than once daily. For a variety of reasons relating to cellular adaptations and safety, this every day approach to dosing may be sub-optimal for managing senescent cells. Instead, an intermittent dosing approach may be the most prudent approach for gradually reducing the burden of lingering senescent cells without disrupting the function of healthy cells.

Therefore, what is needed is a composition that (1) can help manage senescent cells well in advance of age-related disease or pathology, (2) is formulated to include a combination of plant extracts that can overcome tissue heterogeneity to manage senescent cells in a broad range of tissues, (3) targets multiple different SCAPs to overcome the mechanism heterogeneity different populations of senescent cells rely upon, (4) supplies doses of selected plant extracts that are within the range needed to effectively manage senescent cells, and (5) is dosed in an intermittent manner to optimally balance effectiveness and safety. A composition is needed that addresses these multiple dimensions. This need has heretofore remained unsatisfied.

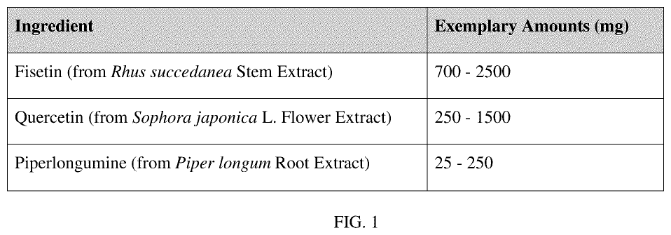

Preferred embodiments of the present invention and their advantages may be understood by referring to FIGS. 1-3. Further features and advantages of the invention, as well as the structure and operation of various embodiments of the invention, are described in detail below. Although the invention is described in the context of a compound, any form may be implemented such as, but not limited to a powder, a pill, or a liquid, or other form suitable for various administrations.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Moreover, the described features, structures, or characteristics of the invention may be combined in any suitable manner in one or more embodiments. It will be apparent to those skilled in the art that various modifications and variations can be made to the present invention without departing from the spirit and scope of the invention. Thus, it is intended that the present invention covers the modifications and variations of this invention provided they come within the scope of the appended claims and their equivalents. Reference will now be made in detail to the preferred embodiments of the invention.

In an exemplary embodiment of the present invention and with reference to FIG. 1, a dietary supplement that improves the management of senescent cells. Active agents were selected for their efficacy and synergism in reducing symptoms of aging, promoting mental function, and enhancing physical performance by managing senescent cells. More particularly, the compositions described herein comprise an effective amount of Fisetin (from Rhus succedanea Stem Extract), Quercetin (from Sophora japonica L. Flower Extract), and Piperlongumine (from Piper longum Root Extract). In some embodiments, the effective amounts of each active agent are selected to result in a synergistic or greater than additive effect. As will be appreciated by a person of skill in the art, the active agents described herein may be formulated in any number of suitable manners for delivery to an individual in need thereof. In a preferred embodiment, the composition is formulated for oral delivery. The individual ingredients, or any combination thereof, may or may not be produced in a manner to be more bioavailable. In one embodiment of the invention, the total mg in one dose is about 2000 mg of total active agents. Optionally, the total mg of active agents in one dose is about 1900 mg, 1800 mg, 1700 mg, 1600 mg, 1500 mg, 1400 mg, 1300 mg, 1200 mg, 1100 mg, or 1000 mg. In one embodiment, the total mg in one dose is 1750 mg total active agents. As can best be seen in FIG. 1, the amount of Fisetin (from Rhus succedanea stem extract) is present in an amount of between about 700-2500 mg, the amount of Quercetin from Sophora japonica L. Flower Extract) is between about 250-1500 mg, and the amount of Piperlongumine (from Piper longum root extract) is between about 25-250 mg of the total amount. In one embodiment, the amount of Fisetin (from Rhus succedanea stem extract) is about 1400 mg, the amount of Quercetin (from Sophora japonica L. Flower Extract) is about 300 mg, and the amount of Piperlongumine (From Piper longum Root Extract) is about 50 mg. The composition can be formulated as capsules or may be formulated for administration in tablets or other delivery form. In a preferred embodiment, the composition described herein is administered in four capsules.

The composition described herein comprises a plurality of botanical active agents for use in managing senescent cells. At least one of the active botanical agents includes an effective amount of Fisetin derived from the stems of Rhus succedanea. Fisetin is a yellow plant pigment found in many plants in low amounts. Rhus succedanea stems are the primary commercial source for making plant extracts standardized for Fisetin. A standardized plant extract is an extract that has one or more components present in a specific, guaranteed amount, usually expressed as a percentage. The intention behind the standardization is to create a plant extract with consistent composition. Rhus succedanea stems undergo extensive raw material sourcing and manufacturing process controls, including extracting and concentrating processes, to produce an extract with a guaranteed amount of Fisetin. Fisetin is among the most promising compounds for managing senescent cells. The word senolytic is used to describe compounds that target SCAP or non-SCAP cellular mechanisms and cause lingering senescent cells to proceed through apoptosis. Senomorphic (or senostatic) is used to describe compounds that alter the phenotype of senescent cells (phenotype has to do with structural and functional characteristics) typically in ways that counter SASP and prevent secondary senescence (i . . . e, the bystander effect). Fisetin has had both senolytic and senomorphic activity in studies in a variety of tissues, including adipose tissue (Romashkan et al., 2021; Yousefzadeh et al., 2018).

The composition as described herein further includes an effective amount of quercetin. Quercetin is a yellow plant pigment found in many plants in low amounts. Sophora japonica flower buds are the primary commercial source for making plant extracts standardized for quercetin. Similar to Fisetin, quercetin is among the most promising compounds for managing senescent cells and has been both senolytic and senomorphic in studies. Quercetin has been more active in non-adipose tissues such as bone marrow and endothelial cells (Xing et al., 2022; Y. Zhu et al., 2015) so, combined with Fisetin, offers broader tissue support.

Also included in the disclosed composition is an effective amount of piperlongumine. Piperlongumine is an alkaloid found in several species of pepper plants, most notably long pepper (Piper longum) from which it got its name. It is found in higher amounts in the roots than the fruits. Piperlongumine has shown senolytic activity (Wang et al., 2016). Because piperlongumine acts through some different mechanism than Fisetin and quercetin to manage senescent cells (X. Liu et al., 2018; Zhang et al., 2018), it complements these other plant extracts in the composition.

The composition works unexpectedly well in that by using these select, multiple ingredients that support managing senescent cells in a variety of different tissues, with ingredients targeting both overlapping and distinct SCAPs, the composition has a significantly more robust effect than the individual plant extracts on their own. The constituents form a synergistic compound where the combined effect is greater than the sum of their separate effects. The intermittent dosing balances effectiveness and safety.

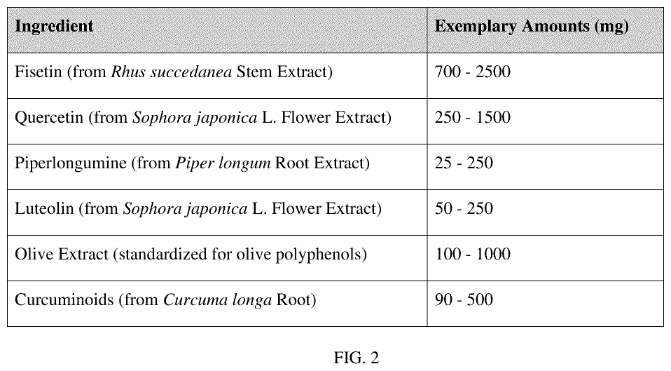

In an exemplary embodiment of the present invention and with reference to FIG. 2, the composition further comprises one or more of the following ingredients: Luteolin (from Sophora japonica L. Flower Extract), Olive Extract (standardized for olive polyphenols), Curcuminoids, such as curcumin (from Curcuma longa Root). The individual ingredients, or any combination thereof, may or may not be produced in a manner to be more bioavailable. In one embodiment of the invention, the total mg in one dose is between about 1250 mg to about 6500 mg of active agents as can best be seen in FIG. 2. In one embodiment, the total amount of Fisetin (from Rhus succedanea stem extract) is between about 700 mg to 2500 mg. In another embodiment, the amount of Fisetin per dose is between about 1000 mg to 2300 mg. In still another embodiment, the amount of Fisetin is about 2250 mg. Advantageously, the composition is formulated for delivery in a plurality of capsules. In one embodiment, a preferred dosage comprises the administration of four capsules, each capsule comprising about 550 mg/capsule of active ingredients. In a particularly preferred embodiment, each capsule comprises 560 mg/capsule of active agents. The total amount of Quercetin is between about 250-1500 mg, with at least one embodiment comprising about 300 mg of Quercetin derived from Sophora japonica L. Flower Extract. Piperlongumine, derived from Piper Longum Root Extract, is present in an amount of between 50-250 mg, preferably around 50 mg in at least one embodiment. The composition may further include Luteolin (from Sophora japonica L. Flower Extract) in an amount between about 25-250 mg of luteolin. In one embodiment, the amount of luteolin is about 150 mg. The formulation can further include olive extract, standardized for olive polyphenols, in an amount of between about 100-1000 mg per dose. In one embodiment, the amount of olive extract is about 250 mg. The formulation can also include an effective, synergistic amount of curcuminoids, in an amount of between about 90-500 mg. In one embodiment, the amount of curcuminoids (from Curcuma longa root) is present in an amount of about 92 mg.

Luteolin is a yellow plant pigment found in many plants in low amounts. Sophora japonica flower buds are the primary commercial source for making plant extracts standardized for luteolin. Luteolin has had senolytic activity (Vázquez et al., 2022; Yousefzadeh et al., 2018). Olives are one of the foods found in the Mediterranean diet. Some of the health-promoting properties of both the Mediterranean diet, and of olives, may be related to their polyphenols. Olive oils, fruits, and leaves are all sources of polyphenols, but differ in the relative amounts of certain polyphenols. Oleuropein is found in higher amounts in leaf extracts, while another type of polyphenol, hydroxytyrosol, is in higher amounts in fruit extracts. Oleuropein may help manage senescent chondrocytes in cartilage of joints and has senomorphic action (i.e., helps prevent secondary) (Frediani et al., 2022; Menicacci et al., 2017; Varela-Eirín et al., 2020). Curcumin comes from turmeric (Curcuma longa roots) and is the pigment that gives turmeric its characteristic yellow-orange color. Curcumin helps manage senescent cells in intervertebral disc cells (Cherif et al., 2019), which adds to the variety of tissues being supported by the composition. And, curcumin is synergistic with other ingredients in the composition including Fisetin, quercetin (Barra et al., 2022), and milk thistle (Abdel-Magied & Elkady, 2019).

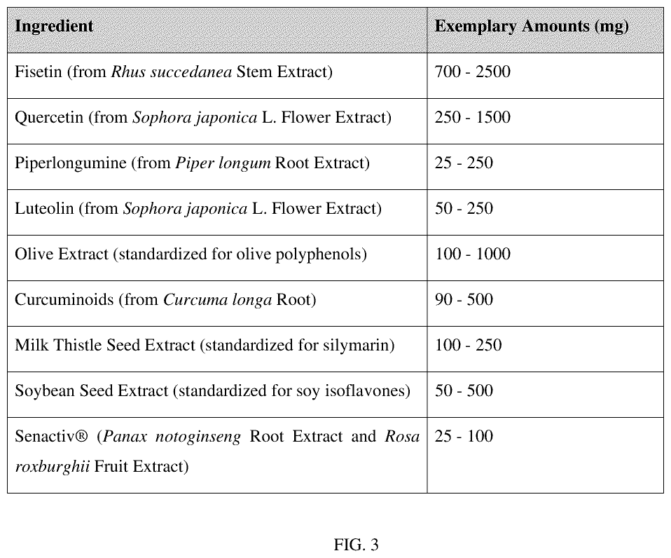

In an exemplary embodiment of the present invention and with reference to FIG. 3, the composition can further include one or more of the following ingredients: Milk Thistle Seed Extract standardized for silymarin, Soybean Extract standardized for soy isoflavones, and Senactiv® (a proprietary combination of Panax notoginseng Root and Rosa roxburghii Fruit Extracts, NuLivScience, Brea, CA). The individual ingredients, or any combination thereof, may or may not be produced in a manner to be more bioavailable. In one embodiment of the invention, the composition described herein comprises active ingredients in ranges described in FIG. 3. In some embodiments, the total mg in one dose comprises between about 1350 mg to about 6900 mg active agents. In another embodiment, the amount of active agents is about 2500 mg. 2600 mg, 2700 mg. 2800 mg, 2900 mg, or 3000 mg. In an embodiment, the active agents are formulated to include about 2600 mg of active agents in a plurality of capsules, for example it may be formulated for ingestion in six capsules, each capsule comprising between about 400 and 500 mg of active agents/capsule.

The composition as detailed in FIG. 3 can include between 700 and 2500 mg of Fisetin from Rhus succedanea stem extract. In one embodiment, the amount of Fisetin is about 1400 mg. The composition further includes Quercetin from Sophora japonica L Flower in an amount of between about 250-1500 mg. In one embodiment the amount of Quercetin is about 300 mg. Piperlongumine from Piper longum root extract is present in an amount of between about 25-250 mg. In an embodiment, the amount of piperlongumine is about 50 mg. The composition can further include luteolin (from Sophora japonica L. Flower Extract) in an amount of between 50-250 mg, with 150 mg being the preferred amount of Luteolin. Further ingredients include Olive Extract (in an amount of between about 100-1000 mg, with 250 mg being a preferred amount), curcuminoids from Curcuma longa Root (in an amount of between 90-500 mg, preferably around 90 mg of active ingredient), Milk Thistle Seed Extract standardized for silymarin in an amount of between about 100-250 mg, preferably around 125 mg, soybean seed extract (standardized for soy isoflavones) in an amount of between about 50-500 (preferably about 200 mg), and Senactive® senolytic supplement (NuLivScience, Brea, CA), in an amount of between about 25-100 mg, preferably around 50 mg.

Milk Thistle Seed Extract is made from the seed-like fruits of Silybum marianum. They have been used for more than 2,000 years in Europe and countries bordering the Mediterranean sea and have been one of, if not the most studied liver tonic herbs. The main active substance in milk thistle is silymarin, which is a complex mixture of flavonolignan compounds including silybin (often called silibinin in scientific studies). Silybin may help manage senescent cells (B. Liu et al., 2019) and would be expected to be synergistic with some other ingredients in the composition including curcumin (Abdel-Magied & Elkady, 2019). Soybean is native to East Asia, where it is a staple food. Soybeans are the most common source of isoflavones in human food; the major isoflavones in soybean are genistein, daidzein, and glycitein. Because of this, cuisines that consistently consume fermented soybean foods have much higher isoflavone intake than Western diets. Soy isoflavones, daidzein as an example, have been identified as candidates for helping to manage some types of senescent cells (Kusumoto et al., 2021). Notoginseng root (Panax notoginseng) and sweet chestnut rose (Rosa roxburghii) have a long history of use in Traditional Chinese Medicine. P. notoginseng is rich in bioactive compounds common to plants in the ginseng family, namely polysaccharides and saponins, with Ginsenoside Rg1 being one of the most abundant ginsenosides in notoginseng. R. roxburghii is native to the southwest region of China. It is known for its high vitamin C, superoxide dismutase (SOD), and strong antioxidant properties. Senactiv® is a patented combination of P. notoginseng and R. roxburghii that has helped manage senescence cells after exercise (Wu et al., 2019) and would be expected to diversify the tissue support of the composition to also include managing senescent cells in muscle tissue.

The present composition may be administered orally. In such an embodiment, the senescent cell management support aid may be integrated into a liquid, capsule, tablet, chewable tablet, or pill. In an exemplary embodiment, the composition is integrated into capsules and taken intermittently orally with breakfast and/or lunch. The intermittent dosing consists of taking the composition for a couple to a few days, followed by a week to several weeks where the composition is not taken. This intermittent dosing protocol is then repeated.

In addition to the exemplary ingredients disclosed, other ingredients may be used to assist administration. In an example where the senescent cell management aid is integrated into a capsule or tablet, the composition may also include binding agents or other inactive ingredients.

The present composition may be administered orally. In such an embodiment, the senescent cell management aid may be integrated into a liquid, capsule, tablet, chewable tablet, or pill. In an exemplary embodiment, the composition is integrated into capsules and taken intermittently orally with breakfast and/or lunch. The intermittent dosing consists of taking the composition for a couple to a few days, followed by a week to several weeks where the composition is not taken. This intermittent dosing protocol is then repeated. In such an embodiment, a dietary supplement to improve mental function and physical performance by managing senescent cells may be prepared by first combining Rhus succedanea stem extract standardized for Fisetin, Piper longum root extract standardized for Piperlongumine, and Sophora japonica L. flower extract standardized for Quercetin (as exemplary ingredients) and mixing until a homogeneous mixture is produced. The composition may then be integrated into capsules to be taken orally.

The present disclosure may also be administered to a person in other ways without departing from the embodiments contemplated herein including: pressing into a tablet form, including in a food or beverage, and other related methods of oral ingestion.

To make the composition, one takes the ingredients as described in the exemplary embodiments and mixes them in powder form until a homogeneous mixture is achieved. The composition is then packaged into capsules to be taken orally.

The compounds of the invention may be administered orally in appropriate dosage units, as desired. The compounds of the invention may be in a powder form, liquid form or a combination of powder and liquid forms. For oral administration, the compounds may be provided as a tablet, aqueous or oral suspension, dispersible powder or granule, emulsion, hard or soft capsule, syrup or elixir. Compositions intended for oral use may be prepared according to any method known in the art for the manufacture of dietary supplement compositions and such compositions may contain one or more of the following agents: sweeteners, flavoring agents, coloring agents, preservatives, solubilizers, wetting agents, stabilizers, colorants, antioxidants, coating agents and diluents. The sweetening agents and flavoring agents will increase the palatability of the preparation. Tablets containing Fisetin, quercetin, and piperlongumine in admixture with non-toxic pharmaceutically acceptable excipients suitable for tablet manufacture are acceptable. Such excipients include inert diluents such as calcium carbonate, sodium carbonate, lactose, calcium phosphate or sodium phosphate; granulating and disintegrating agents such as corn starch or alginic acid; binding agents such as starch, gelatin or acacia; and lubricating agents such as magnesium stearate, stearic acid or talc. Tablets may be uncoated or may be coated by known techniques to delay disintegration and absorption in the gastrointestinal tract and thereby provide a sustained action over a longer period of time. For example, a time delay material such as glyceryl monostearate or glyceryl distearate alone or with a wax may be employed.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

Aqueous suspensions may contain the compounds of the invention in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions may be formulated by suspending the active ingredient in a vegetable oil, such as arachis oil, olive oil, sesame oil or coconut oil, or in a mineral oil such as liquid paraffin. The oil suspension may contain a thickening agent, such as beeswax, hard paraffin or cetyl alcohol. Sweetening agents, such as those set forth above, and flavoring agents may be added to provide a palatable oral preparation. These compositions may be preserved by an added antioxidant such as ascorbic acid. Dispersible powders and granules of the invention suitable for preparation of an aqueous suspension by the addition of water provide the active ingredient in admixture with a dispersing or wetting agent, a suspending agent and one or more preservatives. Additional excipients, for example sweetening, flavoring and coloring agents, may also be present. Syrups and elixirs may be formulated with sweetening agents such as glycerol, sorbitol or sucrose. Such formulations may also include a demulcent, a preservative, a flavoring or a coloring agent. For assistance in formulating the compositions of the present invention, one may refer to Remington's Pharmaceutical Sciences, 15th Edition, Mack Publishing Co., Easton, Pa.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

### EXAMPLES

The present invention is described below with reference to Examples 1 and 2.

### Example 1: Changes in RAND SF-36 Scores

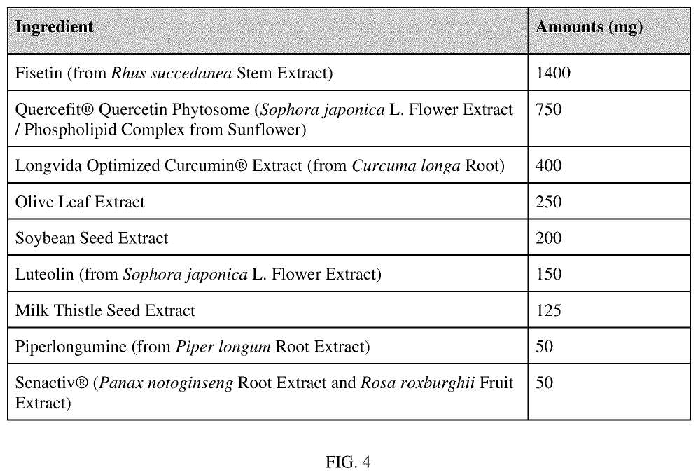

In order to confirm the mental function and physical performance benefits in humans, capsules of an exemplary embodiment of the composition were formed. Details of capsule contents are shown in FIG. 4.

The 36-Item Short Form Survey Instrument (SF-36) was developed at RAND as part of the Medical Outcomes Study. It is one of the most widely used tools to assess health status and health-related quality of life. The SF-36 scores eight health concepts: (1) General Health, (2) Vitality (i.e., energy and fatigue levels), (3) Mental Health (i.e., psychological well-being) (4) Emotional Health Limitations (i.e., to what degree emotional health limits work or daily activities), (5) Physical Health Limitations (i.e., to what degree physical health limits work or daily activities), (6) Social Functioning (i.e., ability to engage in normal social activities), (7) Physical Functioning (i.e., ability to perform physical activities), and (8) Bodily Comfort.

Nineteen adults ranging in age from 29 to 58 years old completed the RAND SF-36 prior to and following six doses of the composition shown in FIG. 4.

Participants took six capsules of the composition for two consecutive days, followed by twelve days with no supplementation. This two-day dosing cycle was repeated three times. Because of this intermittent dosing schedule, participants took the composition for a total of six days over about one month.

Respective scores in seven of the eight concepts assessed by the RAND SF-36 were higher, and therefore improved after three dosing cycles of the composition shown in FIG. 4. Greatest improvements from baseline levels for the group of participants occurred with: (1) Emotional Health Limitations (78% improvement), (2) Physical Health Limitations (42% improvement), (3) and Vitality (38% improvement). There were also improvements in Social Functioning (23%), General Health (21%), Mental Health (17%), and Bodily Comfort (8%). The administration of the composition described herein resulted in demonstrative improvement in abating symptoms associated with aging.

### Example 2: Changes in WOMAC Scores

In order to confirm the mental function and physical performance benefits in humans, capsules of an exemplary embodiment of the composition were formed. Details of capsule contents are shown in FIG. 4.

The Western Ontario and McMaster Universities Arthritis Index (WOMAC) is widely used in the evaluation of Hip and Knee Osteoarthritis. It is a self-administered questionnaire consisting of 24 items divided into 3 subscales relating to pain, stiffness, and physical limitations.

Ten adults ranging in age from 32 to 66 years old completed the WOMAC prior to and following six doses of the composition shown in FIG. 4.

Participants took six capsules of the composition for two consecutive days, followed by twelve days with no supplementation. This two-day dosing cycle was repeated three times. Because of this intermittent dosing schedule, participants took the composition for a total of six days over about one month.

Respective scores in all three of the subscales assessed by the WOMAC were lower, and therefore improved after three dosing cycles of the composition shown in FIG. 4. Improvements from baseline levels for the group of participants were: (1) Pain scores decreased by 53%, (2) Stiffness scores decreased by 53%, (3) and Physical limitations decreased by 48%.

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

1. A composition for managing senescent cells, comprising: an effective amount of Fisetin, piperlongumine, and quercetin.

2. The composition of claim 1, wherein said Fisetin is derived from Rhus succedanea stem extract.

3. The composition of claim 1, wherein said piperlongumine is derived from Piper longum root extract.

4. The composition of claim 1, wherein said quercetin is derived from Sophora japonica L. flower extract.

5. The composition of claim 1, further comprising an effective amount of a botanical agent selected from the group consisting of luteolin sourced from Sophora japonica L. flower extract, curcuminoid is sourced from Curcuma longa root, polyphenols sourced from olive leaf extract, and combinations thereof.

6. The composition of claim 1, further comprising an effective amount of a compound selected from the group consisting of silymarin, soy isoflavones, Senactive® senolytic supplement (NuLivScience, Brea, CA), and combinations thereof.

7. The composition of claim 6, wherein said silymarin is sourced from milk thistle seed extract.

8. The composition of claim 6, wherein said soy isoflavones are sourced from soybean extracts.

9. The composition of claim 1, wherein the composition is integrated into a capsule, tablet, chewable tablet, or pill for oral delivery.

10. The composition of claim 9, wherein the composition is formulated for a prescribed administration protocol.

11. The composition of claim 10, wherein the administration protocol comprises administration of said composition once daily for a period of at least two days but not more than four days followed by a period of at least twelve days but no more than thirteen weeks without treatment, with the administration repeated following this same dosing protocol.

12. The composition of claim 1, wherein the composition is integrated into a liquid or powder.

13. The composition of claim 1, wherein the effective amount of Fisetin is between about 700 mg to 2500 mg per dose.

14. The composition of claim 13, wherein the effective amount of Fisetin is 1400 mg.

15. The composition of claim 1, wherein the effective amount of quercetin is between about 250 mg and 1500 mg per dose.

16. A method for improving mental function and physical performance associated with aging, comprising:
administering to an individual in need thereof a composition comprising:
an effective amount of Rhus succedanea stem extract;
an effective amount of Piper longum root extract; and
an effective amount of Sophora japonica L. flower extract;
wherein the administration of said composition manages senescent cells.

17. The method of claim 16, wherein the composition further comprises Luteolin, curcuminoids, olive polyphenols, and combinations thereof.

18. The method of claim 17, wherein the composition further comprises Milk Thistle Seed extract, Soybean extracts, Panax notoginseng root extract, Rosa roxburghii fruit extract, and combinations thereof.

19. The method of claim 16, wherein the method comprises orally administering said composition in the form of a capsule, tablet, chewable tablet, liquid, powder, or pill.

20. The method of claim 16, wherein the composition is administered once a day for a period of two days followed by a period of four weeks without administration of said composition.
